(19) United States
(12) Patent Application Publication
(10) Pub. No.: US 2004/0266749 A1
(43) Pub. Date: Dec. 30, 2004

(54) Methods for cystic fibrosis disease assessment and methods for treating anaerobic P. aeruginosa biofilms in cystic fibrosis disease

(76) Inventors: Daniel J. Hassett, Cincinnati, OH (US); George M. Hilliard, Hernando, MS (US); San-Sun Yoon, Cincinnati, OH (US)

Correspondence Address: DINSMORE & SHOHL, LLP, 1900 CHEMED CENTER, 255 EAST FIFTH STREET, CINCINNATI, OH, 45202, US

(21) Appl. No.: 10/689,327

(22) Filed: Oct. 20, 2003

Related U.S. Application Data
(60) Provisional application No. 60/419,612, filed on Oct. 18, 2002.

(51) Int. Cl. G01N 33/53; G01N 33/567; G01N 33/554; G01N 33/569; A61K 31/545; A61K 31/407

(52) U.S. Cl. 514/200; 514/210.09; 435/7.2; 435/7.32

(57) ABSTRACT

Methods for Cystic Fibrosis disease assessment in an individual comprise detecting the presence or absence of outer membrane protein in a sample from an individual or the methods comprise detecting the presence or absence of outer membrane protein antibodies in a sample from an individual. Methods for treating anaerobic Pseudomonas aeruginosa biofilms in Cystic Fibrosis disease comprise detecting the presence of outer membrane protein in a sample from an individual; and selecting a therapy regimen for the individual based on the presence of OprF, wherein the anaerobic Pseudomonas aeruginosa biofilms in Cystic Fibrosis disease are treated by the therapy regimen or the methods comprise detecting the presence of outer membrane protein antibodies in a sample from an individual; and selecting a therapy regimen for the individual based on the presence of OprF antibodies; wherein the anaerobic Pseudomonas aeruginosa biofilms in Cystic Fibrosis disease are treated by the therapy regimen.

### FIELD OF THE INVENTION

The present invention is directed toward methods for Cystic Fibrosis (CF) disease assessment in an individual. The present invention is also directed toward methods for treating anaerobic P. aeruginosa biofilms in Cystic Fibrosis disease in an individual.

### BACKGROUND OF THE INVENTION

Cystic Fibrosis (CF) is one of the most common fatal genetic disorders in the United States. CF is most prevalent in the Caucasian population and occurs on an average of one in every 3,300 live births. A mutation in a gene that encodes a chloride channel—the cystic fibrosis transmembrane conductance regulator—produces partially functional or completely dysfunctional channels. Depending on where the gene is mutated and on whether the person carries one or two copies of the mutated allele, the prognosis varies widely: heterozygous individuals are fine for life; those who are homozygous for the mutation get CF; and if patients have the most common CF allele—DF508—they typically die at the age of 31.

CF patients develop thick mucus secretions resulting from disruption of the salt/water balance. They clog bronchial tubes in the lungs and plug exit passages of the pancreas and intestines, leading to loss of function of these organs. And it is in this thick mucus—depleted of oxygen by the metabolic activity of aerobic bacteria, neutrophils, and even epithelial cells—where P. aeruginosa thrives.

Pseudomonas aeruginosa is an important opportunistic pathogen that infects the immunocompromised, elderly, cancer chemotherapy patients, and individual suffering from the inherited autosomal disease, cystic fibrosis (CF). In CF lung disease, P. aeruginosa is trapped in thickened, dehydrated, hypoxic mucus lining in airway epithelia. Moreover, morphologic data suggests that the airway lumen of cystic fibrosis (CF) patients harbor P. aeruginosa biofilms that are characterized as spherical microcolonies.

As chronic lung infection of CF patients by P. aeruginosa is the leading cause of morbidity and mortality associated with the diseases, it would be advantageous to develop a method for assessing the Cystic Fibrosis disease in an individual. It also would be advantageous to develop treatment strategies for anaerobic P. aeruginosa biofilms in Cystic Fibrosis (CF) disease.

### SUMMARY OF THE INVENTION

Accordingly, it is an object of the invention to provide methods for Cystic Fibrosis disease assessment in an individual. It is a further object of the invention to provide methods for treating anaerobic Pseudomonas aeruginosa biofilms in Cystic Fibrosis (CF) disease in an individual.

In accordance with one aspect of the invention, methods for Cystic Fibrosis (CF) disease assessment in an individual are provided. The methods comprise detecting the presence or absence of outer membrane protein (OprF) in a sample from an individual.

In accordance with another aspect of the invention, methods for Cystic Fibrosis (CF) disease assessment in an individual are provided. The methods comprise detecting the presence or absence of outer membrane protein (OprF) antibodies in a sample from an individual. In accordance with yet another aspect of the invention, methods for treating anaerobic Pseudomonas aeruginosa biofilms in Cystic Fibrosis (CF) disease in an individual are provided. The methods comprise the steps of detecting the presence of outer membrane protein (OprF) in a sample from an individual; and selecting a therapy regimen for the individual based on the presence of OprF. The anaerobic Pseudomonas aeruginosa biofilms in Cystic Fibrosis (CF) disease are treated by the therapy regimen.

In accordance with yet another aspect of the invention methods for treating anaerobic Pseudomonas aeruginosa biofilms in Cystic Fibrosis (CF) disease in an individual are provided. The methods comprise the steps of detecting the presence of outer membrane protein (OprF) antibodies in a sample from an individual; and selecting a therapy regimen for the individual based on the presence of OprF antibodies. The anaerobic Pseudomonas aeruginosa biofilms in Cystic Fibrosis (CF) disease are treated by the therapy regimen.

Additional embodiments, objects and advantages of the invention will become more fully apparent in view of the following detailed description.

### DETAILED DESCRIPTION OF THE DRAWINGS

The following detailed description will be more fully understood in view of the drawings comprising:

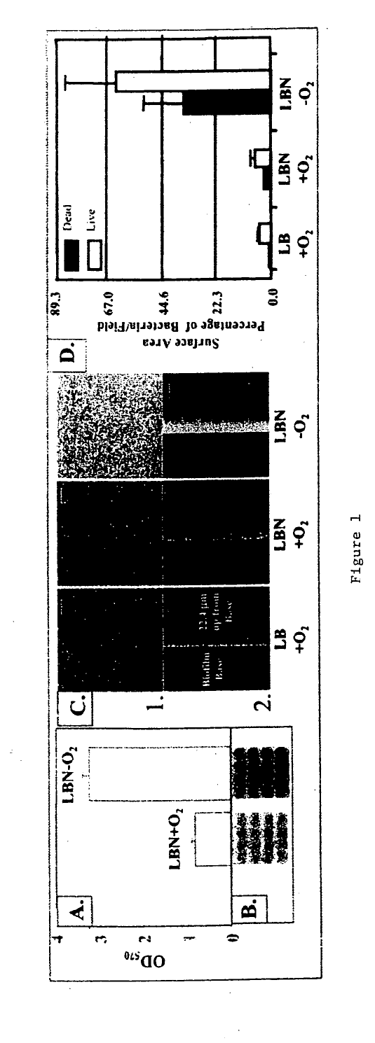

FIG. 1 is an illustration of the influence of oxygen on P. aeruginosa biofilm formation. (A) Quantitative differences between aerobic and anaerobic biofilms after crystal violet solubilization with ethanol. Bars represent the x+/−SE of the crystal violet optical density at 570 nm (n=16 wells); (B) An overnight culture of wild-type P. aeruginosa PAO1 (˜2×10⁷ CFU) is used to inoculate LBN under aerobic or anaerobic conditions in microtiter dishes. Following incubation at 37° C. for 24 hr, biofilms are stained with 1% crystal violet and photographed; (C) Confocal laser microscopic analysis of aerobic and anaerobic P. aeruginosa PAO1 biofilms. Live cells are stained with syto-9 (green) and dead cells with propidium iodide (red). The top (1) and sagittal views (2) are projected from a stack of 56 images taken at 0.4 μm intervals for a total of 22.4 μm. LB, L-broth; LBN, L-broth with 1% KNO₃; D. Quantification of biofilm cell viability. The Y-axis of the graph measures the proportion of the microscope field occupied by live (white bars) versus dead (black bars) bacteria the. Six fields were measured and expressed as mean and standard deviation;

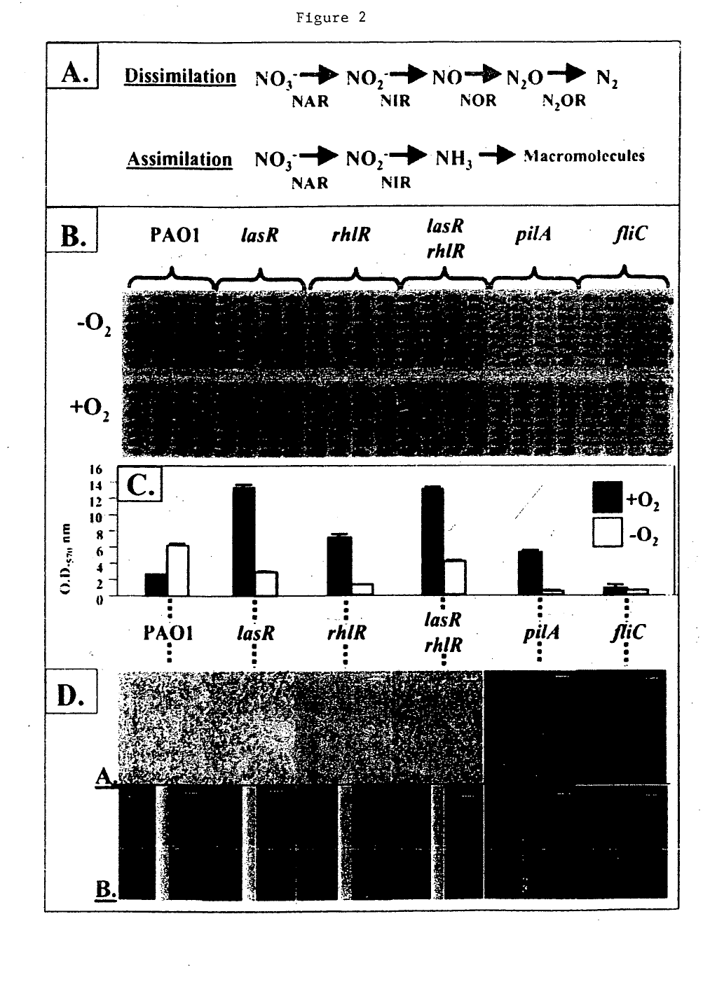

FIG. 2 is an illustration of the influence of oxygen on biofilm formation by isogenic P. aeruginosa mutants known to be defective in aerobic biofilm formation. (A) P. aeruginosa pathways for dissimilatory and assimilatory nitrate reduction. NAR, nitrate reductase; NIR, nitrite reductase; NOR, nitric oxide reductase; N₂OR, nitrous oxide reductase; (B) Crystal violet staining of wild-type and isogenic mutants of P. aeruginosa grown as biofilms under aerobic (+O₂) or anaerobic (−O₂) conditions in LBN; (C) Quantitative differences between aerobic (black bars) and anaerobic (white bars) biofilms; (D) Confocal laser microscopic analysis of anaerobic P. aeruginosa biofilms. Genotypes are given above or between each panel or panel set. A, top view; B, sagittal view;

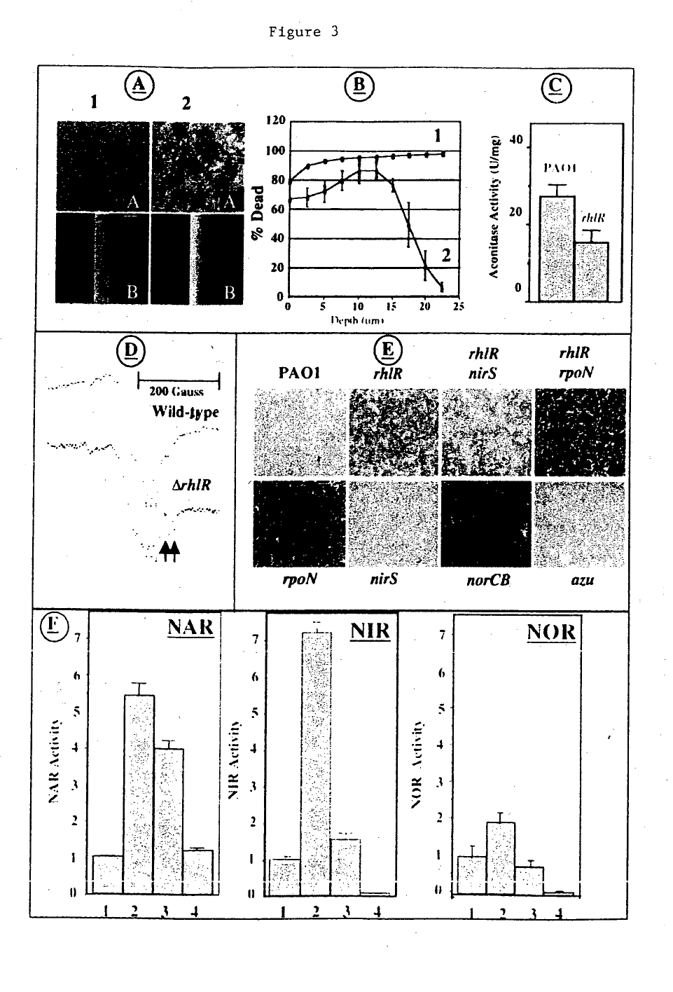

FIG. 3 is an illustration of the overproduction of NO by ΔrhlR bacteria account for anaerobic cell death. (A) P. aeruginosa rhlR mutant bacteria are grown as biofilms under anaerobic conditions as described in FIG. 2D and top and sagittal images are captured by scanning confocal laser scanning microscopy. Panels 1A,B, ΔrhlR; panels 2A,B, ΔrhlR+5 mM carboxy-PTIO; (B) Quantitative assessment of biofilm viability from the sagittal sectioning data gleaned in panels 1 and 2. Line 1, ΔrhlR; line 2, ΔrhlR+5 mM carboxy-PTIO; (C) Anaerobic aconitase activity (x+/−SE, n=3); (D) EPR spectra of anaerobic PAO1 and ΔrhlR bacteria. The arrows denote the NO—Fe—S cluster signals in rhlR mutant bacteria; (E) Anaerobic biofilms are grown and top view confocal images are collected. The strain designation is given above or below each panel; (F) NO₃— (NAR), NO₂— (NIR) and NO(NOR) reductase activity in stationary phase cell extracts of anaerobically grown bacteria. The relative level of wild-type NAR, NIR and NOR activity is assigned a value of 1 and used as a comparison with activities of various test strains. Lane 1, PAO1; lane 2, rhlR; lane 3, rhlRrpoN; lane 4, nirS;

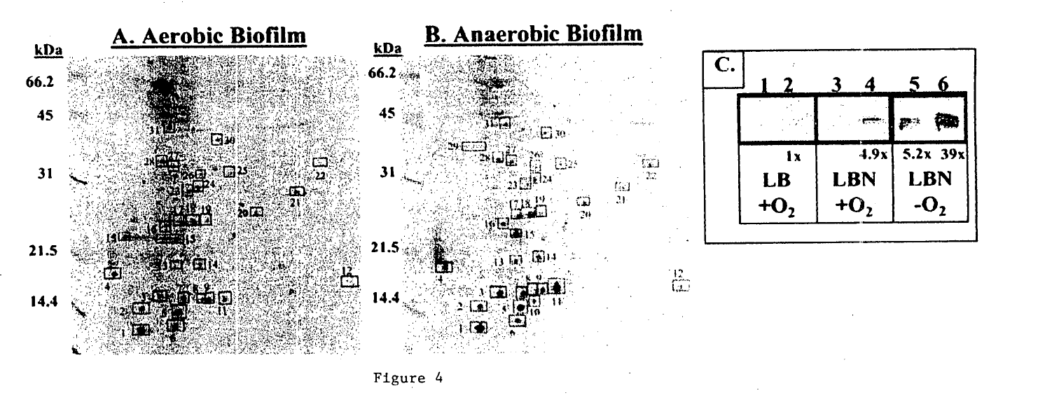

FIG. 4 is an illustration of two-dimensional SDS polyacrylamide gel of aerobic and anaerobic biofilm P. aeruginosa proteins. P. aeruginosa PAO1 is grown in microtiter dishes containing aerobic LBN (A) or anaerobic LBN (B) for 24 h at 37° C. Biofilm bacteria are harvested at 4° C. and are poured over crushed ice to prevent new protein synthesis. Whole cell extracts from aerobic and anaerobic biofilm samples are separated via 2-dimensional gel electrophoresis and stained with silver nitrate. The relative intensity of boxed spots in each 2-D gel is quantified by Melanie 3.0 2-D imaging software (see Table 1); (C) Relative abundance of OprF in three different in vitro cultures; aerobic L-broth biofilm (lane 1, 2), aerobic LBN biofilm (lane 3, 4), anaerobic LBN biofilm (lane 5, 6). Lanes 1, 3 and 5 contain 0.5 μg protein. Lanes 2, 4 and 6 contain 5 μg protein. Lane 2 serves as the aerobic control for quantification, a value set at IX;

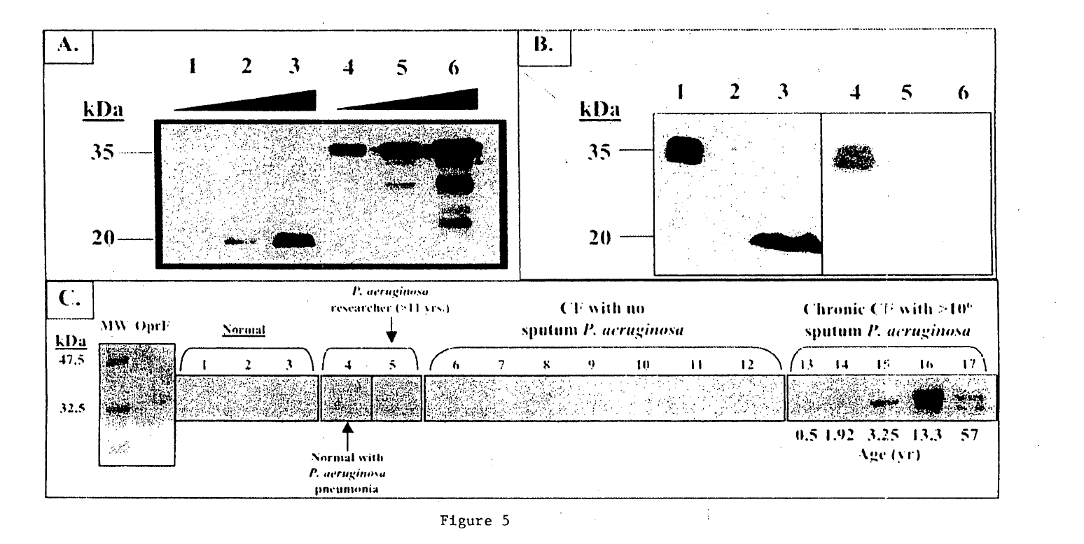

FIG. 5 is an illustration of the identification of OprF in CF lung secretions and production of anti-OprF antibodies by chronically infected CF patients. (A) Comparison of OprF present in secretions from a chronically infected CF patient with the amount of OprF produced in anaerobically grown P. aeruginosa. Lanes 1-3 CF lung secretions from a chronically infected transplant patient; lanes 4-6 contain whole cell proteins from PAO1 grown anaerobically in LBN. Lane 1,4, 100 ng; lane 2,5, 1 μg; lane 3,6, 10 μg; (B) Western blot analysis of OprF production using wild-type (lanes 1,4), oprF mutant (lane 2,5), and airway secretions (lanes 3,6). The antibody used for lanes 1-3 is derived against a linear N-terminal OprF epitope, while that used to probe lanes 4-6 is derived against a C-terminal epitope; (C) Purified recombinant P. aeruginosa OprF is blotted onto PVDF membranes prior to western analysis using serum from, normal patients, normal individuals with P. aeruginosa pneumonia, CF patients with no sputum P. aeruginosa, or chronically infected CF patients with >10⁶ sputum P. aeruginosa/ml. Sera from normal at age 0.83 yr (lane 1), 2 yr (lane 2), 2.25 yr (lane 3); Sera from normal individuals with P. aeruginosa pneumonia (lane 4: 2.68 yr with >10⁶ Staphylococcus aureus and >106 P. aeruginosa, lane 5: corresponding author who has worked with P. aeruginosa over 11 years). Sera from CF patients with no sputum P. aeruginosa at age: (lane 6: 11.1 yr, lane 7: 2.5 yr with 10⁵ Hemophilus influenzae and 103 Streptococcus pneumoniae, lane 8: 0.1 yr with 2×10³ airway oropharyngeal flora, lane 9: 3.75 yr with 4×10⁴ airway oropharyngeal flora, lane 10: 0.16 yr with 4×10³ airway oropharyngeal flora, lane 11: 3.5 yr with 1×10⁴ airway oropharyngeal flora, lane 12: 6.83 yr with 1×10³ airway oropharyngeal flora). Sera from chronically infected CF patients with >10⁶ P. aeruginosa in sputum (lane 13: 0.5 yr with 5×10⁶ P. aeruginosa, lane 14: 3.25 yr with 1×10⁶ P. aeruginosa, lane 15: unknown age with 2×10⁷ P. aeruginosa, lane 16: 13.25 yr with >1×10⁶ P. aeruginosa, lane 17: 57 yr, ΔF508/R117H);

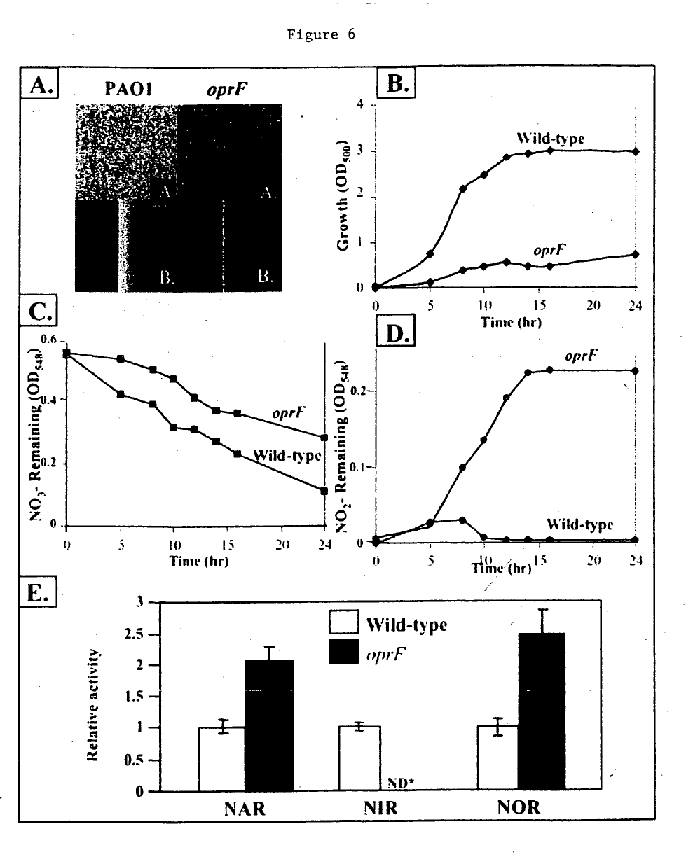

FIG. 6 is an illustration of the role of P. aeruginosa oprF in anaerobic growth, biofilm, and dissimilation nitrate reduction. (A) is Top and sagittal images of wild-type PAO1 and oprF mutant bacteria grown as anaerobic biofilms; (B) Anaerobic growth curves of wild-type and oprF mutant bacteria; (C) Nitrate uptake of wild-type and oprF mutant bacteria; (D) Nitrite levels in culture media of wild-type and oprF mutant bacteria; (E) Anaerobic NAR, NIR and NOR activity in wild-type (white bars) and oprF mutant bacteria (black bars) (n=3, x+/−SE). ND=not detected.

### DETAILED DESCRIPTION OF THE INVENTION

Pseudomonas aeruginosa is an important opportunistic pathogen that is trapped in a thickened, dehydrated, hypoxic mucus lining in airway epithelia of a Cystic Fibrosis (CF) lung. The biochemical and genetic mechanisms utilized by P. aeruginosa to form biofilms have been elucidated in vitro using aerobic flow-through systems, which have emphasized the role of quorum sensing (QS) pathways. In addition, chemical studies have revealed that CF sputum contains two QS signaling molecules, N-butyryl-L-homoserine lactone (C₄-HSL) and N-(3-oxododecanoyl)-L-homoserine lactone (3O—C₁₂-HSL), at a 3:1 ratio of C₄-HSL to 3O—C₁₂-HSL. Both signaling molecules participate in QS signaling processes that involve two master transcriptional activator pairs, LasR/3O—C₁₂-HSL and RhlR/C4-HSL, respectively. The in vitro formation of aerobic P. aeruginosa biofilms on abiotic substrata is dependent, in part, upon the LasR/3O—C₁₂-HSL QS system and the presence of flagella and type IV pili.

In contrast to aerobic in vitro biofilms, P. aeruginosa biofilms in the CF lung grow in stagnant mucus, and recent data have revealed that this environment is anaerobic and favors production of the viscous exopolysaccharide, alginate. P. aeruginosa is also capable of planktonic (detached) growth via anaerobic or aerobic respiration. Nitrate (NO₃—), nitrite (NO₂—), and nitrous oxide (N₂O) are terminal electron acceptors that support anaerobic respiration. The sequential 8-electron reduction of NO₃— to N₂ is also called denitrification. Sufficient NO₃— has been measured in CF airway surface liquid and CF sputum to permit growth of P. aeruginosa under anaerobic planktonic conditions.

An initiating event in the pathogenesis of CF lung disease is mucus stasis. It has been demonstrated that steep hypoxic gradients are present in stationary mucus plaques from CF airway cultures. With the onset of bacterial infection, oxygen consumption by bacteria, neutrophils and CF airway cells renders the thick mucus essentially anaerobic.

Accordingly, the inventors have discovered that P. aeruginosa forms robust anaerobic biofilms and their survival require the rhl quorum sensing circuit and nitric oxide reductase. In addition, the inventors have discovered through Proteomic analyses that anaerobic P. aeruginosa biofilms reveal an outer membrane protein, OprF, whose expression is upregulated ˜40-fold. Analyses of CF mucus also detected OprF and further, CF patients exhibited anti-OprF antisera.

The rhl QS Circuit and Anaerobic P. aeruginosa Infections in CF Airway Disease.

A 3:1 ratio of C₄— to 3O—C₁₂-HSL has been measured in in vitro biofilms and in CF sputa. While not wishing to be bound by theory, the inventors believe that the RhlR-C₄-HSL tandem is important for survival of bacteria during anaerobic CF lung disease as their studies show that bacteria lacking RhlR die via metabolic NO suicide because of 5- and 7-fold dysregulatory increases in NAR and NIR activities, respectively. The small increase (2-fold) in potentially protective NOR activity in the rhlR mutant cannot provide relief from such toxic NO levels. Thus, while not wishing to be bound by theory, the inventors believe that the rhl QS circuit, and, in particular, critical components related to NO₃— mediated anaerobic respiration, may be targets for killing of anaerobic biofilm P. aeruginosa in CF lung disease.

Other Mutations that May Support Anaerobic Survival of P. aeruginosa in Chronically Infected CF Airways.

In contrast to the rapid death of anaerobic rhlR mutant bacteria, anaerobic rhlRnirS, rhlRrpoN, nirS, rpoN, and azu mutant biofilms are mostly alive. The unifying feature of each of these mutants is that they have little or no anaerobic NIR activity, and thus generate low NO levels. Virtually all strains of the study, with the exception of a nor CB mutant, are capable of robust anaerobic growth with NO₃— as a terminal electron acceptor. Thus, in the case of a rhlR mutant lacking NIR or RpoN, there is sufficient ATP production from NO₃— reduction to NO₂— without conversion to potentially toxic NO.

In CF, sputum isolates frequently possess rpoN mutant phenotypes, including a lack of flagella and type IV pili. In fact, it has been shown that 39% of sputum isolates from 1030 chronically infected CF patients lacked flagella and pilus-mediated motility, and many were complemented by multiple copies of the rpoN gene. While not wishing to be bound by theory, it is postulated that mutants lacking RpoN may have a survival advantage because they resist nonopsonic phagocytosis and also conserve energy. In addition, rpoN mutant bacteria produce markedly reduced NIR activity, which protects them from NO poisoning.

OprF, an Outer Membrane Protein Critical for Optimal Anaerobic Growth that is also Produced in Abundance During Chronic CF Lung Disease.

The inventors have discovered that the outer membrane porin, OprF, is only detectable in anaerobic biofilms based upon the sensitivity of 2-D gels and MALDI-TOF proteomic identification technologies. Importantly, the inventors also detected OprF in secretions harvested from freshly excised lungs of CF patients. The inventors also discovered that OprF antibodies are raised by CF patients that are chronically infected with P. aeruginosa, suggesting, while not wishing to be bound by theory, that OprF is constitutively expressed during the course of CF lung disease.

The importance of OprF in anaerobic growth is revealed by the oprF mutant exhibiting (i) a dramatically impaired anaerobic growth rate and final cell density relative to wild-type bacteria and (ii) a complete absence of NIR activity. While not wishing to be bound by theory, the inventors believe that there are two potential scenarios for the precise mechanism connecting OprF to an absence of NIR activity. First, because OprF has been shown to be associated with peptidoglycan, a destabilized peptidoglycan in an oprF mutant may compromise cellular integrity, making cells highly fragile. This, in turn, may lead to leakage of periplasmic proteins into the extracellular milieu. Yet, since there would still be little or no NO₂— in the supernatant, a more likely explanation is that OprF may interact directly with NIR, allowing for a stabilization of enzymatic activity.

CF lung disease has been shown to dramatically worsen when P. aeruginosa converts to the mucoid, alginate-overproducing form. OprF has also been detected in mucoid but not nonmucoid bacteria, yet the precise connection between OprF and mucoidy has been unclear. When mucoid bacteria are grown under static aerobic conditions, these organisms revert to a nonmucoid, antibiotic- and phagocyte-susceptible form. When grown anaerobically, it has been shown that this does not occur.

The inventors have discovered that increased OprF expression and persistence of mucoid P. aeruginosa in CF provide additional evidence that the mucus lining the airways, especially in chronically infected CF patients, is anaerobic. Importantly, it should be noted that the efficacy of many antibiotics such as the “front-line” CF aminoglycoside, tobramycin, is significantly reduced or ineffective under anaerobic conditions. The discovery of impaired anaerobic growth, NO₃— uptake, and an absence of NIR in the oprF mutant suggest that OprF may provide an opportunity for developing a successful therapeutic strategy for combating anaerobic P. aeruginosa biofilms in CF lung disease. Since OprF has been successfully used as protein and DNA vaccines in a mouse model of P. aeruginosa chronic lung infection, it is likely that anti-OprF antibodies may offer some protection during early CF airway disease.

Based upon the inventors discoveries, as set forth in detail above, the inventors have further discovered methods for Cystic Fibrosis (CF) disease assessment in an individual which comprise detecting the presence or absence of outer membrane protein (OprF) in a sample from an individual. The inventors have also discovered methods for Cystic Fibrosis (CF) disease assessment in an individual which comprise detecting the presence or absence of outer membrane protein (OprF) antibodies in a sample from an individual.

In addition, the inventors have discovered methods for treating anaerobic P. aeruginosa biofilms in Cystic Fibrosis (CF) disease in an individual which comprise the steps of detecting the presence of outer membrane protein (OprF) in a sample from an individual; and selecting a therapy regimen for the individual based on the presence of OprF. The anaerobic P. aeruginosa biofilms in Cystic Fibrosis (CF) disease are treated by the therapy regimen. The inventors have further discovered methods for treating anaerobic P. aeruginosa biofilms in Cystic Fibrosis (CF) disease in an individual are provided which comprise the steps of detecting the presence of outer membrane protein (OprF) antibodies in a sample from an individual; and selecting a therapy regimen for the individual based on the presence of OprF antibodies. The anaerobic P. aeruginosa biofilms in Cystic Fibrosis (CF) disease are treated by the therapy regimen.

As used herein, “individual” is intended to refer to a human, including but not limited to, children and adults. One skilled in the art will recognize the various samples available for detecting the presence or absence of a outer membrane protein (OprF) in an individual, any of which may be used herein. Samples include, but are not limited to, airway surface liquid, sputa, or combinations thereof. Moreover, one skilled in the art will recognize the various samples available for detecting the presence or absence of a outer membrane protein (OprF) antibodies in an individual, any of which may be used herein. Samples include, but are not limited to, blood samples, tissue samples, body fluids, or combinations thereof.

As used herein, “assessment” is intended to refer to the prognosis, monitoring, delaying progression, delaying early death, staging, predicting progression, predicting response to therapy regimen, tailoring response to a therapy regimen, of Cystic Fibrosis disease based upon the presence or absence of outer membrane protein (OprF) or OprF antibodies in an individual's sample.

One skilled in the art will appreciate the various known direct and/or indirect techniques for detecting the presence or absence of outer membrane protein, any of which may be used herein. These techniques include, but are not limited to, amino acid sequencing, antibodies, Western blots, 2-dimensional gel electrophoresis, immunohistochemistry, autoradiography, or combinations thereof.

As used herein, “therapy regimen” is intended to refer to a procedure for delaying progression, or delaying early death associated with Cystic Fibrosis disease and/or Pseudomonas aeruginosa in a Cystic Fibrosis individual. In one embodiment, the therapy regimen comprises administration of agonists and/or antagonists of outer membrane protein. In another embodiment, the therapy regimen comprises agonists and/or antagonists of Pseudomonas aeruginosa. In a further embodiment, the therapy regimen comprises agonists and/or antagonists of outer membrane protein (OprF).

In conclusion, the inventors have discovered that P. aeruginosa forms better biofilms while growing during anaerobic NO₃— respiration. The most efficient form of this mode of growth requires respiration via NO₃— reduction and tight control of NO levels during this process. Successful therapy of P. aeruginosa in CF lung infections should be directed at anaerobic biofilms and pathways necessary for dissimilatory NO₃— reduction and to modulate production of (rhl QS system) and removal of NO(NOR). The inventors further discovered that OprF is yet a second potential drug target. Anaerobic conditions trigger a marked increase in OprF expression and promote or maintain the mucoid, alginate-overproducing phenotype in CF patients or in vitro, respectively. Thus, agents that enhance mucus clearance coupled with the development of new antibiotics that are effective under anaerobic conditions may be required to enhance killing of P. aeruginosa during chronic CF lung infections.

The detailed description will be more fully understood in view of the examples.

### EXAMPLES

Throughout the examples, the following experimental procedures may be referenced:

Bacteria

All bacteria are derivatives of P. aeruginosa PAO1 (Holloway, 1969). Allelic exchange are used for deletion or insertion mutagenesis. Bacteria are grown in either Luria-Bertani (L)-broth or L-broth containing 1% KNO₃ (LBN).

Growth of Biofilms.

(a) Microtiter dish method. Polystyrene microtiter dishes containing 100 μl of L-broth or LBN/well are inoculated with 5 μl of ten-fold diluted and optical density adjusted overnight culture and plates are allowed to incubate at 37° C. under aerobic and anaerobic conditions for 24 hr. Anaerobic growth is achieved in an anaerobic chamber (Form a). Crystal violet staining and quantification of biofilms is performed as previously described (O'Toole and Kolter, 1998).

(b) Confocal examination of biofilms. For biofilm architecture examination, circular coverslips are glued to the bottom of 35×10 mm polystyrene tissue culture dishes with small holes in the base (Falcon). The plates are sterilized overnight by UV irradiation. Three ml of aerobic L-broth, aerobic LBN or anaerobic LBN is inoculated with ˜10⁷ CFU of overnight L-broth grown culture. After 24 hr at 37° C., biofilms are washed with saline and stained with 0.5 ml of a LIVE/DEAD BacLight bacterial viability stain (Molecular Probes, Inc., Eugene, Oreg.). Images are acquired on a Zeiss LSM 510 laser scanning confocal unit attached to an Axiovert microscope using a 63×1.4 NA oil immersion objective. For two color images, samples are scanned sequentially at 488 nm and 546 nm. Syto 9 (green fluorescence) is detected through a 505-530 nm bandpass filter and propidium iodine (red fluorescence) is detected through a 560 nm longpass filter and presented in two channels of a 512×512 pixel, 8-bit image.

Construction of a P. aeruginosa Tryptic Library Database.

All 5,570 translated open reading frames from the P. aeruginosa genome (www.pseudomonas.com/maps/map1.htm) are downloaded into ProFound (http://prowl.rockefeller.edu/cgi-bin/ProFound) and a tryptic fragment library is assembled.

Experiments Designed to Monitor NO Overproduction During Anaerobic Growth of rhlR Mutant Bacteria.

(a) Anaerobic biofilm protection by 2-(4-Carboxyphenyl)-4,4,5,5-tetramethylimidazoline-1-oxyl-3-oxide (carboxy-PTIO). Biofilms of rhlR mutant bacteria are grown anaerobically in the presence vs. absence of 5 mM carboxy-PTIO (Molecular Probes, Eugene, Oreg.), an NO scavenger.

(b) Aconitase activity as an indirect measure of NO poisoning. Cell extracts from anaerobic bacteria are prepared and assayed for aconitase activity (Gardner and Fridovich, 1991).

(c) Electron paramagnetic resonance spectroscopy of the dinitrosyl-iron-dithiol complex after anaerobic growth of P. aeruginosa. Wild-type and rhlR bacteria are grown for 48 hr under anaerobic conditions with shaking at 100 rpm. Samples are concentrated 10-fold and aliquots are removed for assessment of turbidity and total cell dry weight. Equivalent turbid slurries of each organism are drawn into 1 ml tuberculin syringes that are immersed in a dewer of liquid nitrogen. The tip of the frozen syringe is then cut off and the slurry allowed to partially thaw so that the frozen contents could slide out freely into a new dewer of liquid nitrogen. The frozen bacterial slivers are placed into an EPR cuvette that was kept at −180° C. by liquid helium. Disassembly of the [3Fe-4S] clusters of the inactive forms of P. aeruginosa proteins upon the anaerobic production of NO is accompanied by the formation of two characteristic Fe—S—NO species. These are examined spectroscopically using a Bruker EMX EPR. EPR settings were: 77K, 20 mW, MA, 4.0; 2.6 TC.

(d) NO₃— (NAR), NO₂— (NIR) and NO reductase (NOR) activity assays. NAR activity is measured in cell extracts of stationary phase anaerobic LBN-grown bacteria (Lester and DeMoss, 1971). For NIR and NOR activity assays, the disappearance of NO₂— and NO is followed using the Griess reagent (Nims, 1995) and a NO electrode (Gardner et al., 1998), respectively.

Two-Dimensional Gel Electrophoresis and MALDI-TOF Mass Spectrometric Protein Analyses.

Bacteria are grown in microtiter dishes under aerobic and anaerobic conditions. After 24 hr at 37° C., planktonic bacteria are removed and the saline-washed biofilms are detached by scraping the wells. Cell extracts are prepared from cultures harvested by centrifugation at 10,000×g for 5 min at 4° C. Bacteria are washed twice in 10 mM Tris-HCl, pH 8.0, and after three freeze-thaw cycles, sonicated on ice. Cell debris is clarified by centrifugation at 13,000×g for 10 min at 4° C. Protein is estimated by the method of Bradford (Bradford, 1976). Immobiline Drystrips (Amersham) are used for separation of proteins in the first dimension. The strips are equilibrated in SDS-PAGE buffer and separated by 12% SDS-PAGE in the second dimension. Mass spectrometric protein identification is performed as previously described ((Shevchenko et al., 1996), and http://proteomics.uc.edu). Protein spots are excised from 2-D silver-stained polyacrylamide gels (http://proteomics.uc.edu/ Silver %20Stain.htm). Quantification of protein spots in 2-D gels is performed using Melanie 3.0 imaging software (Swiss Institute of Bioinformatics). Protein spots are digitized and quantified on a volume basis by mathematical integration of optical density over spot area.

### Example 1

Robust P. aeruginosa Biofilm Formation During Anaerobic vs. Aerobic Conditions.

The conditions selected for this study mimic the static growth mode characteristic of biofilms in immobile mucus plaques within CF airways and contrast to “flow” biofilms that better represent urinary tract, bloodstream, or catheter biofilm models. A simple, and highly reproducible microtiter dish biofilm assay is employed (O'Toole and Kolter, 1998) to assess whether (i) P. aeruginosa could form biofilms under strict anaerobic vs. aerobic conditions and (ii) how these biofilms differ using parameters that included thickness, density and viability. As shown in FIGS. 1A and B, P. aeruginosa biofilms formed during anaerobic growth are greater than 3-fold larger (i.e., more bacteria) than during aerobic conditions.

Next, to determine the spatial architecture of anaerobic versus aerobic biofilms, biofilms are grown on circular glass coverslips and visualized by confocal laser scanning microscopy both from top-to-bottom and sagittal views. As shown in FIG. 1C, aerobic biofilms (LB, +O₂) are characterized by typical microcolony formation with more bacteria at the biofilm base. The addition of NO₃— to the culture media stimulate slightly more bacteria to adhere to the glass surface and, again, the biofilm bacteria to be more concentrated at the base (LBN, +O₂). In contrast, a thick, compact biofilm is formed by anaerobic bacteria (LBN, −O₂). Using a vital stain and confocal microscopy, the inventors observe that anaerobic biofilms contained ˜1.8-fold greater live (green) vs. dead (red) organisms, a trait similar to aerobic biofilms (FIG. 1D). Despite the fact that anaerobic biofilms on glass are greater than when grown on plastic (˜7- vs. 3-fold, compare FIGS. 1C,D vs. A,B), the inventors results confirm that P. aeruginosa prefers the anaerobic biofilm mode of growth.

### Example 2

Which Gene Products are Required for Anaerobic as Compared with Aerobic Biofilms?

Because P. aeruginosa appears to be growing as anaerobic biofilms in CF airways (Worlitzsch et al., 2002), the inventors compare selected gene products required for aerobic biofilms with those required for optimal anaerobic biofilm formation. As a reminder (FIG. 2A), anaerobic respiration in P. aeruginosa (also called respiratory NO₃— reduction or denitrification) involves the sequential 8-electron reduction of NO₃— to N₂. In contrast, assimilatory NO₃— reduction involves uptake of NO₃— and its reduction to NH₃. Also, recall that the in vitro formation of aerobic P. aeruginosa biofilms using aerobic flow-through systems is dependent, in part, upon the LasR/3O—C₁₂-HSL QS tandem (Davies et al., 1998) and the presence of flagella and type IV pili (O'Toole and Kolter, 1998).

To test whether these gene products are required for anaerobic biofilm formation under static conditions, wild-type and isogenic lasR, rhlR, lasRrhlR, pilA (pilus-deficient), and fliC (flagellum-deficient) mutants are grown under aerobic vs. anaerobic conditions. As shown in FIGS. 2B and C, the lasR, rhlR, lasRrhlR and pilA mutants form very robust biofilms under aerobic conditions, even better than wild-type bacteria. In contrast, none of the mutants could form anaerobic biofilms as well as wild-type bacteria. The fliC mutant produce poor biofilms regardless of whether oxygen was present or not. While not wishing to be bound by theory, the inventors believe that This is likely because twitching motility, mediated by type IV pili, is reduced significantly in the fliC mutant during aerobic growth in the presence of NO₃— and absent during anaerobic growth (data not shown). Consistent with this observation, one of the mutants reveal a pathway that is absolutely required for anaerobic biofilm formation. The pilA mutant could form a good aerobic biofilm, presumably mediated by flagella, but form poor anaerobic biofilms, suggesting that type IV pili are critical for anaerobic biofilm formation.

### Example 3

Gene Products Essential for Optimal Viability in Anaerobic Biofilms.

Next, the inventors examine the role of LasR, RhlR, type IV pili, and flagella on anaerobic biofilm formation and cell viability on glass coverslips using confocal laser scanning microscopy (FIG. 2D, parts A [top view], B, [sagittal view]). Consistent with the results discussed above, isogenic pilA and fliC mutants form very poor anaerobic biofilms relative to that formed by wild-type bacteria. In contrast, lasR, rhlR, and lasRrh/R mutants form good biofilms but the number of dead bacteria increased significantly in these mutants. Specifically, under anaerobic biofilm conditions, lasR and, to a greater extent, rhlR and lasRrhlR mutants are nearly all dead when evaluated by confocal laser quantification from the base (˜20-30% alive) to the top of the biofilm (˜99% dead). Because impairment of QS leads to a rapid killing of anaerobic biofilm bacteria, the inventors next pursue experiments designed to elucidate the mechanism of premature cell death.

### Example 4

Death of Anaerobic QS Mutants in a Biofilm is Due to Metabolic Intoxication by Nitric Oxide (NO).

During denitrification, P. aeruginosa produces three gases including NO, N₂O (nitrous oxide) and N₂ (nitrogen gas), with NO being a powerful antimicrobial agent (Fang, 1997). While not wishing to be bound by theory, the inventors believe that death of lasR, rhlR, and lasRrhlR mutants in anaerobic biofilms reflect the relative concentration of NO produced by these organisms. To test this notion, a series of complimentary assays are employed. First, when the rhlR mutant is grown anaerobically in a biofilm, most bacteria are dead, except for some viable organisms (˜20%) that are attached to the biofilm base (FIG. 3A, panel 1). This result is consistent with the anaerobic rhlR mutant biofilm viability data provided in FIG. 2D. The addition of 5 mM carboxy-PTIO, a stable nitroxide and potent scavenger of NO (Pfeiffer et al., 1997), protected the rhlR mutant (FIG. 3A, panels 2 vs. 1). The protection conferred by carboxy-PTIO is observed primarily within the top 10-15 μm of the biofilm (FIG. 3B, line 2 vs. 1), a phenomenon likely due to metabolism of this scavenger by bacteria at the biofilm base.

An indirect measure of NO is a decrease in aconitase activity (Kennedy et al., 1997). NO inactivates aconitase by nitrosation of the [4Fe-4S]²⁺ center. As shown in FIG. 3C, aconitase activity in the rhlR mutant was reduced ˜50% relative to wild-type bacteria. Formation of a nitrosyl complex as Fe—S—NO that is detectable by electron paramagnetic resonance spectroscopy is indicative of NO binding to the solvent exposed iron of proteins with [4Fe-4S] ²⁺ centers (Kennedy et al., 1997). In the rhlR mutant, a NO-mediated iron-nitrosyl free radical spectrum was visible (arrows, FIG. 3D), while a similar spectrum is absent in wild-type bacteria, indicating poisoning of proteins containing Fe—S centers.

To further examine the role of NO in anaerobic biofilm formation and cell viability, biofilms are grown in LBN under anaerobic conditions using a panel of mutant strains designed to test genetically whether NO killed the rhlR mutant bacteria. FIG. 3E shows that most of the anaerobic ΔrhlR biofilm bacteria are dead, consistent with the results described above. In contrast, the majority of the rhlRnirS double mutant, that lacks the only enzyme that produces NO in P. aeruginosai (nitrite reductase), are alive. In addition, other mutants deficient in nitrite reductase, RpoN (a sigma factor that controls nirS expression), or a double rhlRrpoN mutant, are also alive. In parallel, a nor CB mutant generated virtually no biofilm under anaerobic conditions, likely because the NO produced during anaerobic growth could not be detoxified, leading to very poor cell growth. A mutant deficient in the blue copper electron carrier azurin (azu) is also tested in this study. Because azurin feeds electrons to NO₂— reductase, the azu mutant should not overproduce NO. Consistent with our focus for a role of NO in this toxicity, the azu mutant forms a robust, viable biofilm during anaerobic growth.

Finally, to assess whether enhanced NO production in the rhlR mutant is directly attributable to a deregulation of denitrifying enzyme activities, NO₃—, NO₂— and NO reductase activities are assayed in anaerobic cell extracts of selected strains. NO₃— reductase (NAR) is increased >5-fold in the rhlR (lane 2) and 4-fold in the rhlRrpoN mutants (lane 3) relative to wild-type bacteria (lane 1, FIG. 3F). Moreover, NO₂— reductase (NIR, converting NO₂— to toxic NO) activity of the rhlR mutant (lane 2) is nearly 7-fold that of wild-type bacteria (lane 1) but only 1.5-fold higher in rhlRrpoN (lane 3). The NIR activity detected in the rhlRrpoN mutant suggests that lacking rhlR plays a role in nirS activation that is RpoN-independent. The nirS mutant (lane 4) produced negligible NIR activity. In contrast to the marked increases in NAR and NIR activity of the rhlR mutant, protective NO reductase (NOR) activity of the rhlR mutant (lane 2) is only 2-fold that of wild-type organisms (lane 1). In contrast, NOR activity in the rhlRrpoN (lane 3) and nirS mutant (lane 4), that have little or no NIR activity, is reduced ˜30% relative to wild-type activity.

Collectively, the data in FIG. 3 demonstrate that deregulation of anaerobic NO₃— respiration in the rhlR mutant, leading to overproduction of NO, is the cause of premature cell death in these organisms. In contrast, anaerobic organisms lacking or possessing reduced NIR activity such as nirS, rpoN, or azu mutants, thrive under such conditions.

### Example 5

Aerobic Versus Anaerobic Biofilm Proteomics: a Link Found to Differences in Overall Biofilm Physiology.

Because biofilm formation during anaerobic growth is more robust than under aerobic conditions, the inventors questioned whether there are proteins expressed that supported the anaerobic biofilm mode of growth. To test this notion, whole cell lysates from biofilm bacteria grown in LBN under aerobic and anaerobic conditions are separated by 2-D gel electrophoresis (FIG. 4, A [aerobic], B [anaerobic]). A total of 240 protein spots are detected in the anaerobic biofilm gel using the default parameter setting and Melanie 3.0 software, while 213 are detected in the aerobic biofilm gel. Thirty-one spots from identical locations are excised from these gels and 26 proteins were identified by MALDI-TOF analyses.

The proteins are listed in Table 1 with their relative expression levels under each condition (horizontal bar graphs to right of table). Of the 31 protein spots excised for analysis, ˜77% showed greater expression during the anaerobic biofilm mode of growth. The iron-cofactored superoxide dismutase (PA4366) is expressed equally under aerobic and anaerobic conditions. Curiously, there is only one protein that revealed a modest, yet reproducible 1.8-fold increase in expression in aerobic relative to anaerobic biofilms. This is TonB, a protein important in iron sensing/acquisition (Takase et al., 2000). Because aerobic biofilms can have anaerobic zones, one interpretation of this finding is that aerobic biofilms contain a mixture of aerobic and anaerobic bacteria, consistent with a previous report (Ventullo and Rowe, 1982). The most highly expressed anaerobic biofilm protein is azurin that is produced at 5.3-fold greater levels than in aerobic biofilms.

Third, and most importantly, two proteins are detected only in anaerobic biofilms. One is the 50S ribosomal protein L9, the expression of which allowed for two forms. Under aerobic and anaerobic conditions, the protein is nearly equally represented by a 15.5 kDa form. However, a unique and smaller form (spot 10) is observed only under anaerobic conditions. Interestingly, the second protein detected only in anaerobic biofilms is OprF, a channel-forming porin that has been shown to be involved in cell shape and growth in low-salt environments (Rawling et al., 1998).

TABLE 1
Analyses of P. aeruginosa Proteins for Identification and Quantification

| Spot | Protein | PA | | | | Quantification |
|---|---|---|---|---|---|---|
| Number | Name | Number | Z Value | MW | pI | (_O2/_O2) |
| 1 | | | | | | 1568/1625 |
| 2 | Thioredoxin | PA5240 | 1.72 | 12 | 4.7 | 478/742 |
| 3 | Probable DNA binding stress protein | PA0962 | 1.88 | 18.4 | 5 | 253/667 |
| 4 | Hypothetical 18.6 kDa protein | AAK15336a | 1.97 | 18.6 | 4.4 | 448/971 |
| 5 | GroES chaperonine | PA2021 | 1.65 | 10.3 | 5.2 | 1038/1116 |
| 6 | ATP synthase epsilon chain | PA5553 | 1.83 | 14.7 | 5.1 | 505/610 |
| 7 | | | | | | 501/655 |
| 8 | 50S ribosomal protein L9 | PA4932 | 2.39 | 15.5 | 5.4 | 261/210 |
| 9 | Nucleoside diphosphate kinase | PA3807 | 2.32 | 15.6 | 5.5 | 512/542 |
| 10 | 50S ribosomal protein L9 | PA4932 | 2.25 | 15.5 | 5.4 | ND/340 |
| 11 | Azurin precursor | PA4922 | 1.4 | 16.1 | 6.4 | 374/1972 |
| 12 | 50S ribosomal protein L10 | PA4272 | 1.69 | 17.6 | 8.9 | 138/350 |
| 13 | Probable thiol peroxidase | PA2532 | 1.9 | 17.4 | 5.2 | 184/420 |
| 14 | Conserved hypothetical protein | PA3309 | 2.36 | 16.5 | 5.5 | 266/689 |
| 15 | Fe cofactored superoxide dismutase | PA4366 | 2.34 | 21.9 | 5.3 | 995/986 |
| 16 | Inorganic pyrophosphatase | PA4031 | 2.21 | 19.4 | 5 | 306/412 |
| 17 | Probable peroxidase | PA3529 | 2.1 | 21.9 | 5.4 | 264/332 |
| 18 | Probable peroxidase | PA3529 | 2.33 | 21.9 | 5.4 | 449/1026 |
| 19 | | | | | | 259/202 |
| 20 | Alkyl hydroperoxide reductase (AhpC) | PA0139 | 1.91 | 21.6 | 5.9 | 209/377 |
| 21 | Probable TonB-dependent receptor | PA5505 | 2.39 | 28.1 | 7.9 | 423/236 |
| 22 | Probable binding protein component of ABC | PA1342 | 2.09 | 35.2 | 8.5 | 91/248 |
| 23 | Arginine/ornithine binding protein AotJ | PA0888 | 2.37 | 29 | 6.6 | 230/197 |
| 24 | Hypothetical protein | PA4495 | 2.14 | 25.1 | 5.8 | 328/302 |
| 25 | | | | | | 138/233 |
| 26 | | | | | | 187/179 |
| 27 | Elongation factor Ts | PA3655 | 1.76 | 30.7 | 5.2 | 232/413 |
| 28 | Electron transfer flavoprotein alpha subunit | PA2951 | 2.01 | 32.3 | 5 | 487/387 |
| 29 | Outer membrane protein OprF | PA1777 | 1.71 | 38.8 | 5 | ND/196 |
| 30 | Alcohol dehydrogenase | PA5427 | 2.22 | 36.3 | 5.6 | 233/258 |
| 31 | Branched chain amino acid transport | PA1074 | 1.86 | 40.1 | 5.6 | 315/423 |

Table 1. Analyses of P. aeruginosa proteins for identification and quantification. 31 reproducibly represented proteins containing at least 1 pmol of protein are selected for mass spectrometric analysis. Twenty-six proteins are identified with significant certainty (Z value >1.2) and are listed with their relative amount given numerically and based upon the default parameter using Melanie 3.0 software. The numbered proteins that are expressed at higher levels during anaerobic biofilm growth are underlined. ^{a}An NCBI gene bank accession number is assigned to this protein, because no PA number is available for this protein in the P. aeruginosa genome database (www.pseudomonas.com).

Because OprF has been reported to be constitutively produced by P. aeruginosa (Price et al., 2001), the inventors next quantify the level of OprF expression in aerobic vs. anaerobic biofilms by a Western blot analysis. FIG. 4C shows that OprF expression is increased 4.9-fold during aerobic biofilm growth with NO₃— (lanes 4 vs. 2). In contrast, OprF expression is dramatically (39-fold) upregulated during anaerobic biofilm growth (FIG. 4C, lanes 5,6). Thus, the relative abundance of OprF expressed during anaerobic biofilm growth explains why it is not detected in aerobic biofilm 2-D gels in the presence of NO₃—.

### Example 6

CF Patients with Chronic P. aeruginosa Infections Have Lung Secretions Harboring OprF and Raise Anti-OprF Antibodies.

Because OprF may be important for anaerobic biofilm formation, the inventors next test whether OprF is expressed in vivo during chronic CF airway disease. The inventors use analyses that (i) link OprF expression directly to our in vitro data and (ii) supplement these data with important evidence that OprF antibodies are raised by CF patients who are chronically infected with P. aeruginosa.

First, to assess whether OprF is produced in CF patients, a Western analysis is performed using the membrane fraction of airway secretions harvested from a lung resected from a chronically infected CF transplant patient with an airway mucus bacterial density of ˜10⁷-10⁸ CFU/ml. FIG. 5A (lanes 1-3) shows that secretions contained a cleaved ˜20 kDa OprF cross-reactive protein. The normal size of OprF is 35 kDa, yet papain or trypsin cleavage results in a 20 kDa fragment containing the N-terminal half of OprF which forms a protease-resistant β-barrel that contributes to the transmembrane pore (Brinkman et al., 2000). As controls, this fragment from the in vivo specimen cross-react with a monoclonal antibody derived from an N-terminal linear OprF peptide (FIG. 5B, lane 3), but not a C-terminal antibody (lane 6). As controls, both antibodies cross-reacted with wild-type (FIG. 5B, lanes 1,4) but not oprF mutant cell extracts (FIG. 5B, lanes 2, 5). Thus, although expression of OprF in CF airway secretions is lower than in in vitro-grown anaerobic biofilms (˜10⁹ CFU/ml), the amount of P. aeruginosa in secretions is estimated to be 1-2 logs less. Thus, expression of OprF in vivo is consistent with that of anaerobic biofilm expression in vitro.

Next, the inventors focus our Western analyses on antibodies to OprF using sera from a broad spectrum of CF vs. normal individuals (FIG. 5C). While not wishing to be bound by theory, the inventors believe that chronically infected CF patients harboring anaerobically growing P. aeruginosa would raise OprF antibodies. Only chronically infected CF patients with >106 P. aeruginosa/ml sputum raised antibodies to OprF (lanes 13-17). Normals (lanes 1-3), a normal with P. aeruginosa pneumonia (lane 4), the corresponding author who has worked with P. aeruginosa for more than 11 years (lane 5), or CF patients that are sputum-negative for P. aeruginosa possessed no OprF antibodies.

### Example 7

Role of OprF in Anaerobic Growth and NO₂— Reductase Activity.

Because OprF is expressed in anaerobic in vitro biofilms and in CF airway mucus, the inventors determined if OprF is required for anaerobic biofilm formation. FIG. 6A shows that the oprF mutant form a very poor anaerobic biofilm relative to that formed by wild-type bacteria. To test whether this defect is a function of impaired anaerobic growth of the oprF mutant, an anaerobic growth curve is performed. FIG. 6B demonstrates that the growth rate of the oprF mutant (g=2.34 h⁻¹) is 1.8-fold slower than that of wild-type bacteria (g=1.3 h⁻¹) and the peak cell density is at least 40-fold less. During anaerobic growth, NO₃— is taken up and released as NO₂—, typically via NarK2-like extrusion pumps. Once NO₃— is low, however, NO₂— can be then used for additional energy. FIG. 6C demonstrates that NO₃— uptake is more rapid in wild-type bacteria than the OprF mutant. This suggests that OprF could be a channel for NO₃—, NO₂— or both molecules, since each can be used as anaerobic terminal electron acceptors.

In addition, although wild-type bacteria consume NO₂— to undetectable levels after 8 hr of growth, NO₂— accumulated in the oprF mutant culture supernatants (FIG. 6D). We then find that the oprF mutant lacked NIR activity (FIG. 6E), but not NAR or NOR activities. This explains why (i) NO₂— accumulated in the medium and (ii) the oprF mutant cannot grow anaerobically using NO₂— (data not shown). While not wishing to be bound by theory, the inventors postulate that OprF stabilizes NO₂-activity. However, because OprF is known to associate with peptidoglycan (Rawling et al., 1998), lacking OprF would destabilize the peptidoglycan layer, possibly allowing for leakage of periplasmic protein, one of which is NIR.

The specific embodiments in the examples described here in are illustrative in nature only and are not intended to be limiting of the claimed methods. Additional embodiments and variations within the scope of the claimed invention will be apparent to those of ordinary skill in the art in view of the present disclosure.

What we claim is:

1. A method for Cystic Fibrosis (CF) disease assessment in an individual, comprising detecting the presence or absence of outer membrane protein (OprF) in a sample from an individual.

2. The method according to claim 1, wherein the sample comprises airway surface liquid, sputa or combinations thereof.

3. A method for Cystic Fibrosis (CF) disease assessment in an individual, comprising detecting the presence or absence of outer membrane protein (OprF) antibodies in a sample from an individual.

4. The method according to claim 1, wherein the sample comprises blood, tissue, body fluids, or combinations thereof.

5. A method for treating anaerobic Pseudomonas aeruginosa biofilms in Cystic Fibrosis (CF) disease in an individual, comprising the steps of:
a. detecting the presence of outer membrane protein (OprF) in a sample from an individual; and
b. selecting a therapy regimen for the individual based on the presence of OprF;
wherein the anaerobic Pseudomonas aeruginosa biofilms in Cystic Fibrosis disease are treated by the therapy regimen.

6. The method according to claim 5, wherein the sample comprises airway surface liquid, sputa or combinations thereof.

7. A method for treating anaerobic Pseudomonas aeruginosa biofilms in Cystic Fibrosis (CF) disease in an individual, comprising the steps of:
a. detecting the presence of outer membrane protein (OprF) antibodies in a sample from an individual; and
b. selecting a therapy regimen for the individual based on the presence of OprF antibodies;
wherein the anaerobic Pseudomonas aeruginosa biofilms in Cystic Fibrosis disease are treated by the therapy regimen.

8. The method according to claim 5, wherein the sample comprises blood, tissue, body fluids, or combinations thereof.
